(19) United States
(12) Patent Application Publication
(10) Pub. No.: US 2007/0213409 A1
(43) Pub. Date: Sep. 13, 2007

(54) USE OF PREPARATIONS FOR SKIN ENZYME PROTECTION

(75) Inventors: Ute Breitenbach, Hamburg (DE); Andreas Schepky, Hamburg (DE); Ursula Holtzmann, Hamburg (DE); Alexander Filbry, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/573,327

(22) PCT Filed: Mar. 24, 2005
(86) PCT No.: PCT/EP05/51394

§ 371 Date: Feb. 6, 2007

(51) Int. Cl. A61K 8/34

(52) U.S. Cl. 514/738

(57) ABSTRACT

A cosmetic preparation which comprises an active complex of panthenol, glycerol and citrate. The mass ratio of panthenol to citrate anion is from 25:1 to 5:1 and the preparation has a pH value of from 4.6 to 5.4 and an SCTE (Stratum Corneum Tryptic Enzyme) value of from 120 to 170.

As a result of external influences such as environment soiling, irritants and cleansing, the skin and its constituents, such as, for example, enzymes, can become damaged and restricted in their function. Cleansing the skin using surfactant-containing formulations should effectively remove surface lipids and dirt from the surface of the skin. The enzymes in the skin should be damaged as little as possible by this cleansing. The (anionic) surfactants and surfactant systems usually used deactivate the enzymes considerably. As a result, important metabolic physiological processes (desquamation etc.) of the skin are adversely affected.

For the purposes of the present specification, skin enzymes are enzymes which are present on the surface of the skin or close to the surface of the skin. Such enzymes may be: hydrolases, such as proteases, esterases, lipases, phosphatases, sulfatases and transglutaminases, but in particular proteases, such as the stratum corneum tryptic enzyme. The most important stratum corneum enzymes known in the literature are indicated in tables 1 and 2 and below.

TABLE 1
Enzymes which degrade desmosomes and contribute to desquamation

| | Site | Reaction | |
|---|---|---|---|
| Enzyme | of activity | (barrier damage) | Literature |
| SCCE | SC (LB) | Cleavage of protein | Lundström, 1991 |
| | | bonds | Suzuki, 1994 |
| | | | Sondell, 1995 |
| | | | Chang-Yi, 1997 |
| Trypsin | SC | Cleavage of protein | Suzuki, 1994 |
| | | bonds ↑ | Chang-Yi, 1997 |
| Cathepsin | SG | Filaggrin degradation | Hara, 1993, |
| | | keratinization aid | Kawada, 1997 |
| Thiol protease | SC | | Yokozeki, 1987 |

TABLE 2
Enzymes which construct the barriers
and contribute to barrier homeostasis

| | Site of | Reaction | |
|---|---|---|---|
| Enzyme | activity | (barrier damage) | Literature |
| Phospholipase | SG-SC; | Release of fatty acids | Mauro, 1998 |
| A₂₌ | LB | and possibly | Mao-Qiang, 1995 |
| | | cholesterol from | Elias, 1988 |
| | | cholesterol esters | Menon, 1986 |
| Acidic lipase | SC, LB | Release of sterols | Menon, 1986 |
| | | | Elias, 1988 |
| Neutral lipase | SC, LB | Sterol- and fatty acid- | Menon, 1986 |
| | | release | |
| | | Regulation of protein | |
| | | kinases (differentiation) | |
| Sphingomyelinase | SC, LB | Provision of ceramides | Menon, 1986 |
| Ceramidase | SC | Provision of ceramides | Jin, 1994 |
| β-Gluocere- | SC | Conversion of | Holleran, 1992 |
| brosidase | | glycoceramides to | Mauro, 1998 |
| | | ceramides | |
| Steroid | SC | Cholesterol release | Elias, 1988 |
| sulfatase | | from cholesterol sulfate | |
| Sulfatases | SC | Precursor cleavage | Baden, 1980 |

Ammonia lyases play an important role during filaggrin degradation (Kuroda et al., 1979). So too do transglutaminases (Polakowska et al., 1991), which are essential for the formation of the “cornified envelope”. Phosphatases are the hydrolases with the highest overall activity in the stratum corneum. Influence of enzymes on the desquamation (see Schepky et al., 2004, Influence of cleansing on stratum corneum tryptic enzyme (SCTE) in human volunteers, Int. Journal of Cosmetic Science, 26, 245-253)

Rieger writes in 1994 in Cosmetic & Toiletries that the organization of the epidermis requires a chemical modification of constituents of the keratinocytes, inter alia in the lamellar bodies. Elias pointed to the need for hydrolytic (catabolic) enzymes in the skin. Proteases are required for the removal of desmosomal structures. If denaturing surfactants penetrate there and the enzyme activities are considerably impaired, a defective stratum corneum is the result.

To maintain a constant thickness of the stratum corneum, the desquamation rate and the de novo production of the corneocytes must be balanced exactly. Egelrud demonstrated that the proteolysis by proteases is the central event in the desquamation process with the help of a plantar stratum corneum model. The enzymes best characterized with a function during desquamation are the stratum corneum chymotryptic enzyme (SCCE) and stratum corneum tryptic enzyme (SCTE). SCCE has a number of properties which correlate well with its role during desquamation in vivo: the pH profile of its catalytic unit, its specific inhibitor profile and its position in the tissue. SCTE has a similar role to SCCE during desquamation, but must additionally be able to activate inactive SCCE by hydrolysis. It is assumed that this enzyme cleaves autocatalytically from the inactive form to the active form. For both enzymes, it has been shown that topical application of specific inhibitors of these serine proteases (aprotinin and leupeptin) leads to more skin flakes in vivo. Sato et al. reported in 1998 that cholesterol-3 sulfate reduces both the activity of SCCE and also of SCTE through competitive inhibition. This is associated with reduced desquamation. Further proteases (cathepsin D) have been found in the stratum corneum, but are probably responsible primarily for the fine adjustment of the desquamation.

The present invention relates to a cosmetic active complex for skin enzyme protection against the disadvantageous effects of cleansing products.

For the purposes of the present specification, enzyme protection is consequently understood as meaning a significant reduction/reduction in the damage/impairment to the described skin enzymes caused by cleansing. According to the invention this is achieved through care of the skin with formulations which comprise panthenol, glycerol, citrate (active complex) at pH 5. The effect was demonstrated compared to a placebo which does not contain the active complex.

The enzyme protection can be quantified as follows: firstly an ex vivo determination of the effect of surfactants on the trypsin activity in the human epidermis is carried out. For three weeks, test-subjects put cream on either with placebo or verum and, without care, then wash under supervision several times in 3 days using a standard shower product or water on various areas. 24 h later, the upper stratum corneum is extracted. The stratum corneum tryptic enzyme (SCTE) activity in the extract is measured. In parallel, the protein concentration of the extracts is determined in order to obtain the specific trypsin activity (correction for differing extraction of the areas).

Surprisingly, it has been found that a cosmetic preparation comprising an active complex consisting of panthenol, glycerol, citrate, characterized in that the SCTE value of the preparation is between 120 and 170 and the preparation has a pH of from 4.6 to 5.4 and the mass ratio of panthenol to citrate is 25:1 to 5:1, based on the citrate anion, overcomes the disadvantages of the prior art. Such preparations are able to reduce the skin enzyme damage caused by cleansing.

Furthermore, it is preferred according to the invention if the mass ratio of panthenol to glycerol is at least 1:1 to 1:4.

Furthermore, it is preferred according to the invention if the mass ratio of citrate to glycerol is 60:1 to 10:1, based on the citrate anion. The invention also covers the use of the described active complex in a cleansing preparation, adjusted to pH 5, for skin enzyme protection against damage caused by cleansing.

The invention likewise also covers the use of the described active complex in a skincare preparation, adjusted to pH 5, for skin enzyme protection against damage caused by future cleansing.

A method for the cosmetic treatment of the skin comprising at least the steps a) topical application of a preparation which comprises the described active complex, b) cleansing the skin with a preparation comprising surfactants, preferably 5 to 10% lauryl or myreth ether sulfate and 2-8% cocoamidopropylbetaine is also part of this invention.

Likewise, a method for the cosmetic treatment of the skin comprising at least the steps a) topical application of a preparation which comprises the described active complex, b) cleansing the skin with a preparation comprising surfactants, preferably 5 to 10% lauryl or myreth ether sulfate, 2-8% cocoamidopropylbetaine and 1-5% of a further cosurfactant is part of this invention.

Moreover, preparations according to the invention can furthermore comprise substances which absorb UV radiation in the UVB region, where the total amount of the filter substances is, for example, 0.1% by weight to 30% by weight, preferably 0.5 to 10% by weight, in particular 1.0 to 6.0% by weight, based on the total weight of the preparations, in order to provide cosmetic preparations which protect the hair and/or the skin from the entire range of ultraviolet radiation. They can also serve as sunscreens for the hair.

For the purposes of the present invention, advantageous UV-A filter substances are dibenzoylmethane derivatives, in particular 4-(tert-butyl)-4′-methoxydibenzoylmethane (CAS No. 70356-09-1), which is sold by Givaudan under the brand Parsol® 1789 and by Merck under the trade name Eusolex® 9020.

For the purposes of the present invention, advantageous further UV filter substances are sulfonated, water-soluble UV filters, such as, for example:
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer;
salts of 2-phenylbenzimidazole-5-sulfonic acid, such as its sodium, potassium or its triethanolammonium salt, and the sulfonic acid itself with the INCI name Phenylbenzimidazolesulfonic acid (CAS No. 27503-81-7), which is available, for example, under the trade name Eusolex 232 from Merck or under Neo Heliopan Hydro from Haarmann & Reimer;
   advantageous UV filter substances for the purposes of the present invention are also socalled broadband filters, i.e. filter substances which absorb both UV-A and UV-B radiation.

Advantageous broadband filters or UV-B filter substances are, for example, triazine derivatives, such as, for example,
2,4-bis{[4-(2-ethylhexyloxy)-2-hydroxy]phenyl}-6-(4-methoxyphenyl)-1,3,6-triazine (INCI: Aniso Triazine), which is available under the trade name Tinosorb® S from CIBA-Chemikalien GmbH;
tris(2-ethylhexyl) 4,4′,4″-(1,3,5-triazine-2,4,6-triyltriimino)trisbenzoate, also: 2,4,6-tris-[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150.

The other UV filter substances may be oil-soluble or water-soluble.

Advantageous oil-soluble UV-B and/or broadband filter substances for the purposes of the present invention are, for example:
cinnamic acid derivatives, preferably 2-ethylhexyl methoxycinnamate (CAS No.: 5466-77-3), which is available under the trade name Parsol MCX from Givaudan.

The list of specified UV filters which can be used for the purposes of the present invention is not of course intended to be limiting.

Advantageously, the preparations according to the invention comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair and/or the skin from the entire range of ultraviolet radiation.

### EXAMPLES

1) Determination ex vivo of the Effect of Surfactants on the Trypsin Activity in Human Epidermis

To standardize the skin of the test subjects, the test subjects were requested to only use a mild shower gel when washing for three weeks (6% sodium myreth sulfate, 8% sodium cocoamphoacetate). In parallel, the test subjects applied cream twice daily for three weeks to one forearm with example 1 versus the other forearm with examples 2, 3, 4 or 5. The forearms were each divided into two test areas. Care was ended directly prior to the start of the washing process. The test areas were treated for three days in succession, in each case 3 times daily with 1 ml of washing product for 45 s. After the treatment, the test area was rinsed with tap water for 30 s and dried off using a disposable paper towel. On the 1st and 2nd day the areas were treated three times (morning, midday and afternoon), on the 3rd day they were treated twice (morning and midday).

2) Extraction of the Skin Biopsy and Measurement of the SCTE Activity

On the 4th day, SC samples were stripped from the areas by means of a microscope slide coated with sugar solution. Later on, the corneocytes were detached from the microscope slide with PBS buffer and the specific SCTE activity was determined.

3) Stratum Corneum Tryptic Enzyme (SCTE) Activity Assay

100 μl of human skin extract were incubated for 24 h with 150 μl of N-t-BOC-Phe-Ser-Arg-7-amido-4-methylcoumarin (33 μM in PBS; Sigma, St Louis, USA) at 37° C. The SCTE-specific release of fluorescent 7-amino-4-methylcoumarin was ascertained using a fluorescence plate reader (filter ex=360 nm±40, em=460 nm±40 nm, CytoFluor 4000, PerSeptive Biosystems, Framingham, USA).

4) Measurement of the Protein Concentration

In order to calculate the specific trypsin activity of the extracts, the protein content was determined by means of the ninhydrin method following alkaline hydrolysis. The corneocyte solutions were evaporated to dryness and the proteins were hydrolyzed for 5 h at 150° C. with 2 ml of sodium hydroxide solution (6M). The solution was neutralized with 2 ml of hydrochloric acid (6M) and 1 ml of sodium propionic acid buffer (3.35 M, pH 5.5) was added. 50 μl of the lysate were then diluted with 450 μl of double-distilled water and incubated for 20 min at 70° C. with 25 μl of formic acid (0.4% (v/v)) and 500 μl of ninhydrin solution (2% (w/v) ninhydrin in 3.35 M sodium propionic acid buffer with 50% (v/v) ethylene glycol monomethyl ether (Sigma, St Louis, USA)). After cooling, 5 ml of ethanol (50% (v/v) in double-distilled water) were added. The absorption was measured at a wavelength of 570 nm using a spectrophotometer (UVICON 942, Kontron, Milan, Italy) and the corresponding protein concentration was calculated.

5) Formulas

| | Example No. | | | | |
|---|---|---|---|---|---|
| | 1 | | | | |
| | Placebo | 2 | 3 | 4 | 5 |
| Cetearyl Alcohol + | | | 2 | 1 | |
| PEG-40 Castor Oil + | | | | | |
| Sodium Cetearyl Sulfate | | | | | |
| Ceteth-20 + Glyceryl | | | 1.3 | | |
| Stearate | | | | | |
| Lanolin Alcohol | | | 0.2 | | |
| Polyglyceryl-2 | | | | | 3 |
| Dipolyhydroxystearate | | | | | |
| Polyglyceryl-3 | | | | | 2 |
| Diisostearate | | | | | |
| Polyglyceryl-3 | 5 | 5 | | | |
| Methylglucose Distearate | | | | | |
| Sorbitan Stearate | 2 | 2 | 1 | 2 | |
| Diazolidinyl Urea | 0.25 | 0.25 | | | |
| Phenoxyethanol + | 0.5 | 0.5 | 0.5 | 0.5 | 0.5 |
| Methylparaben + | | | | | |
| Ethylparaben + | | | | | |
| Butylparaben + | | | | | |
| Isobutylparaben + | | | | | |
| Propylparaben | | | | | |
| Cera Microcristallina | | | 5 | | |
| Paraffinum Liquidum | | | | 8 | 10 |
| Glycerin | | 5 | 5 | 12 | 10 |
| Cetearyl Ethylhexanoate | | | 0.8 | | |
| Isopropyl Myristate | | | 5 | | |
| Isopropyl Stearate | | | | | 9 |
| Parfum | 0.15 | 0.15 | 0.15 | 0.15 | 0.15 |
| Citric Acid | | 0.1 | 0.1 | 0.1 | 0.1 |
| Diammonium Citrate | | | 0.25 | | |
| Sodium Citrate | | 0.174 | | 0.174 | 0.174 |
| Magnesium sulfate | | | | | 0.6 |
| Cyclomethicone | 5 | 5 | | 3 | |
| Carbomer | | | | 0.25 | |
| Sodium Hydroxide | | | | 0.03 | |
| Cetearyl Alcohol | 2 | 2 | | | |
| Cetyl Alcohol | | | 2.5 | 3 | |
| Cetyl Palmitate | | | 10 | 10 | |
| PEG-150 Distearate | 1 | 1 | | | |
| Panthenol | | 3 | 3 | 5 | 3 |
| Aqua | ad 100 | ad 100 | ad 100 | ad 100 | ad 100 |
| pH | 7 | 5 | 5 | 5 | 5 |
| SCTE standardized | 100 | 155 | 156 | 148 | 125 |
| to placebo = 100 | | | | | |

1-7. (canceled)

8. A cosmetic preparation, wherein the preparation comprises an active complex of panthenol, glycerol and citrate, a mass ratio of panthenol to citrate anion being from 25:1 to 5:1, and wherein the preparation has a pH value of from 4.6 to 5.4 and an SCTE (Stratum Corneum Tryptic Enzyme) value of from 120 to 170.

9. The preparation of claim 8, wherein a mass ratio of panthenol to glycerol is from 1:1 to 1:4.

10. The preparation of claim 8, wherein a mass ratio of citrate anion to glycerol is from 60:1 to 10:1.

11. The preparation of claim 9, wherein a mass ratio of citrate anion to glycerol is from 60:1 to 10:1.

12. A method of protecting skin enzymes against damage caused by cleansing, wherein the method comprises applying to skin the preparation of claim 8, adjusted to pH 5.

13. The method of claim 12, wherein a mass ratio of panthenol to glycerol in the preparation is from 1:1 to 1:4.

14. The method of claim 12, wherein a mass ratio of citrate anion to glycerol in the preparation is from 60:1 to 10:1.

15. A method for the cosmetic treatment of skin, wherein the method comprises
(a) topically applying to skin a composition which comprises an active complex of panthenol, glycerol and citrate, a mass ratio of panthenol to citrate anion in the composition being from 25:1 to 5:1;
(b) cleansing the skin with a preparation which comprises as surfactants from 5% to 15% by weight of at least one of lauryl ether sulfate and myreth ether sulfate and from 2% to 8% by weight of cocoamidopropylbetaine.

16. The method of claim 15, wherein the composition has a pH value of from 4.6 to 5.4 and an SCTE (Stratum Corneum Tryptic Enzyme) value of from 120 to 170.

17. The method of claim 15, wherein a mass ratio of panthenol to glycerol in the composition is from 1:1 to 1:4.

18. The method of claim 15, wherein a mass ratio of citrate anion to glycerol in the composition is from 60:1 to 10:1.

19. The method of claim 17, wherein a mass ratio of citrate anion to glycerol in the composition is from 60:1 to 10:1.

20. The method of claim 15, wherein the preparation further comprises from 1% to 5% by weight of one or more cosurfactants.

21. The method of claim 15, wherein the composition further comprises at least one of a UVA filter substance and a UVB filter substance.

22. A method for the cosmetic treatment of skin, wherein the method comprises
(a) topically applying to skin a composition which comprises an active complex of panthenol, glycerol and citrate, a mass ratio of panthenol to citrate anion being from 25:1 to 5:1, the composition having a pH value of from 4.6 to 5.4 and an SCTE (Stratum Corneum Tryptic Enzyme) value of from 120 to 170;
(b) cleansing the skin with a preparation which comprises as surfactants from 5% to 15% by weight of at least one of lauryl ether sulfate and myreth ether sulfate, from 2% to 8% by weight of cocoamidopropylbetaine and from 1% to 5% by weight of one or more cosurfactants.

23. The method of claim 22, wherein a mass ratio of panthenol to glycerol in the composition is from 1:1 to 1:4.

24. The method of claim 22, wherein a mass ratio of citrate anion to glycerol in the composition is from 60:1 to 10:1.

25. The method of claim 23, wherein a mass ratio of citrate anion to glycerol in the composition is from 60:1 to 10:1.

26. The method of claim 22, wherein the composition further comprises at least one of a UVA filter substance and a UVB filter substance.

27. The method of claim 26, wherein the composition comprises from 0.5% to 10% by weight of the at least one of a UVA filter substance and a UVB filter substance.
